(19) United States
(12) Patent Application Publication
(10) Pub. No.: US 2026/0307053 A1
(43) Pub. Date: Oct. 8, 2026

(54) PHOTOCATALYTIC C-C COUPLING REACTIONS OF TERMINAL ALKYNES USING CUPRIC OXIDE-BASED MIE RESONATOR PHOTOCATALYSTS

(71) Applicant: UNIVERSITY OF SOUTH FLORIDA, Tampa, FL (US)

(72) Inventors: Marimuthu Andiappan, Tampa, FL (US); Dharendra Yogi Goswami, Tampa, FL (US); Shivam Kumar, Tampa, FL (US)

(21) Appl. No.: 19/638,042

(22) Filed: Apr. 2, 2026

Related U.S. Application Data
(60) Provisional application No. 63/782,173, filed on Apr. 2, 2025.

(51) Int. Cl. B01J 23/44; B01J 8/08; B01J 8/10; B01J 23/72; B01J 35/30; B01J 35/33; B01J 35/39; B01J 35/45; C07C 2/84

(52) U.S. Cl. CPC B01J 23/44; B01J 8/085; B01J 8/087; B01J 8/10; B01J 23/72; B01J 35/33; B01J 35/39; B01J 35/395; B01J 35/45; C07C 2/84; B01J 2208/00451; B01J 2208/00867; C07C 2523/44; C07C 2601/16

(57) ABSTRACT

Described herein is a heterogeneous photocatalyst that includes a cupric oxide (CuO) dielectric Mie resonator particle and palladium (Pd) disposed on the CuO in electrical contact. The Pd comprises one or more of nanoclusters, an ultrathin shell, or a porous thin film over at least a portion of the CuO particle. Under broadband illumination and an oxygen-containing atmosphere, the photocatalyst drives oxidative carbon-carbon coupling of terminal alkynes to form diyne products with high selectivity. In representative embodiments, phenylacetylene converts to 1,4-diphenylbutadiyne under illumination, and temperature-matched dark controls distinguish photon-driven electronic contributions from bulk heating. Embodiments include base-free operation and operation in the presence of an inorganic base, reaction media comprising polar aprotic solvents, alcohol solvents, or mixtures, and illumination spanning 300 nm to 2200 nm at intensities from about 10 mW/cm² to about 150 mW/cm².

### CROSS-REFERENCE TO RELATED APPLICATIONS

This application claims priority to U.S. Provisional Patent Application No. 63/782,173 filed Apr. 2, 2025, titled “Photocatalytic C—C Coupling Reactions of Terminal Alkynes using Cupric Oxide-Based Mie Resonator Photocatalysts,” the contents of which are incorporated by reference in their entirety.

### STATEMENT REGARDING FEDERALLY SPONSORED RESEARCH

This invention was made with government support under 2102238, 2102239, and 2237454 awarded by the National Science Foundation. The government has certain rights in the invention.

### FIELD

The subject matter relates to heterogeneous photocatalysis and, in particular, to dielectric Mie-resonant metal oxide photocatalysts and hybrid metal/oxide photocatalysts that drive oxidative carbon-carbon coupling reactions of terminal alkynes under broadband light irradiation.

### BACKGROUND

Carbon-carbon bond-forming reactions support manufacture of pharmaceuticals, agrochemicals, conjugated polymers, and other high-value chemicals. Conventional oxidative coupling and related C—C coupling processes often rely on homogeneous noble-metal catalysts, which are toxic and expensive, and elevated temperatures supplied by fossil-fuel-derived heat, which drives energy consumption and greenhouse-gas emissions.

Light-driven catalysis supplies energy to a catalyst using photons. Photonic excitation produces bulk heating through optical absorption and also produces photoexcited charge carriers that interact with adsorbed reactants on catalyst surfaces. A need exists for robust, scalable heterogeneous catalysts and reactor protocols that quantify photon-driven electronic contributions distinct from heating contributions under comparable temperature conditions.

Plasmonic metal nanostructures have served as light harvesters for certain coupling reactions, yet plasmonic systems dissipate significant energy as heat and sustain excited carriers on short timescales. Dielectric semiconductor nanoparticles engineered to support Mie resonances sustain photoexcited carriers on longer timescales and provide an earth-abundant light-harvesting platform. A further need exists for catalyst architectures that integrate earth-abundant Mie resonators with catalytically active surface sites while maintaining selectivity and stability under oxygen or air.

Potential application areas for the coupling products and related C—C coupling products include pharmaceutical intermediate synthesis, electronic and optoelectronic materials, polymer materials, chemical biology tools, agrochemical intermediates, green-chemistry manufacturing routes that reduce inorganic salt waste through base-free operation, and solar-to-chemical energy storage concepts that convert photon energy into energy-dense molecules.

### SUMMARY

Described herein are hybrid photocatalysts that include a cupric oxide (CuO) dielectric Mie resonator support and palladium (Pd) disposed on the CuO support as nanoclusters and/or an ultrathin shell or a solid or porous thin film. Under broadband illumination and an oxygen-containing atmosphere, the hybrid photocatalyst drives oxidative homocoupling of terminal alkynes to form diynes with high selectivity.

The catalyst operates under base-free conditions while maintaining high conversion, which reduces inorganic salt waste relative to base-assisted oxidative coupling. In other embodiments, the process includes an inorganic base to increase rate while maintaining high selectivity.

Described herein are experimental designs that quantify photon-driven electronic effects distinct from bulk heating. Temperature-matched dark controls isolate an electronic contribution to rate enhancement under illumination.

Optical design guidance via finite-difference time-domain (FDTD) simulations and absorbed-power calculations that relate effective optical size to volume-normalized absorption and spectral overlap with an incident illumination spectrum are also described herein for selecting optimum hybrid photocatalyst size.

### BRIEF DESCRIPTION OF THE DRAWINGS

FIG. 1. Transition from conventional thermal catalysis to hybrid solar light-driven photocatalysis. (A) Fossil-fuel-based thermal processes operate at high temperature and pressure. (B) Purely thermal reactions follow ground-state energy surfaces, requiring high activation energy (Ea). (C) Schematic of solar-driven catalytic technologies, where the reactor is exposed to either solar (S) or concentrated solar (CS) light. For uninterrupted operation, a hybrid system utilizes solar thermal energy during non-illuminated hours. (D) Direct solar light or concentrated solar light exposure enhances reaction rates via localized heating and photoexcited charge carriers that access excited-state pathways. TEI denote transiently excited ions.

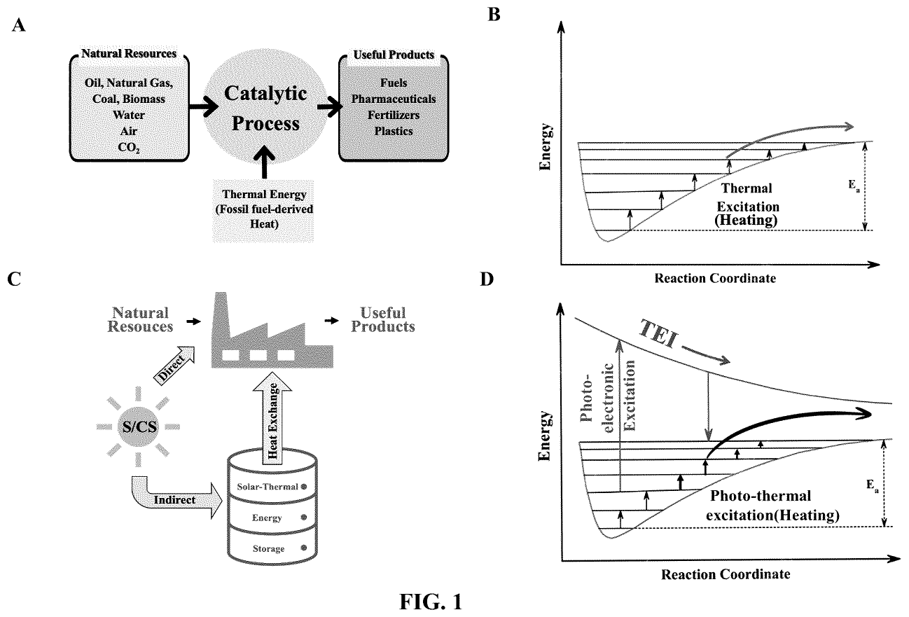

FIG. 2. Structural and optical characterization of CuO—Pd hybrid nanostructures. (A) Shows a SEM image of average particle size of 34±5 nm. (B) Shows a high-angle annular dark-field stem (HAADF-STEM) image of the CuO—Pd hybrid nanocatalysts. (C) Shows energy dispersive X-ray spectroscopy (EDS) elemental mapping of the homogeneous dispersion of Pd throughout the CuO nanoparticles. (D) Shows an EDS map of Cu. (E) Shows an EDS map of O. (F) Shows an EDS map of Pd. D-F demonstrate the uniform spatial distribution of all constituent elements within the hybrid CuO—Pd nanocatalysts.

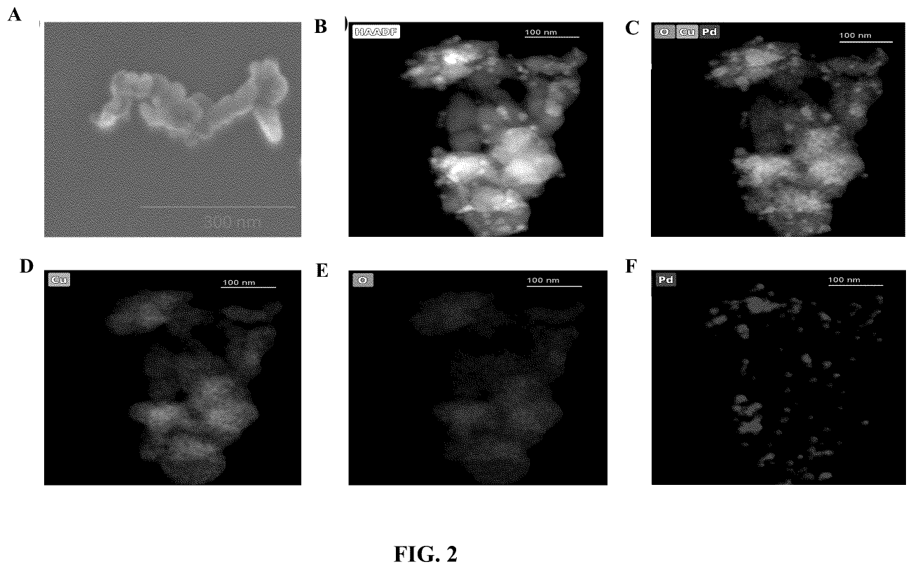

FIG. 3. Structural and FDTD-simulation-based characterization of CuO—Pd hybrid nanostructures. (A) XRD pattern of CuO—Pd material. (B) UV-Vis extinction spectrum of the hybrid nanostructures in the reaction medium, showing characteristic dielectric Mie resonance features. (C) FDTD-simulated, volume-normalized absorption cross sections for CuO—Pd nanospheres (40-400 nm in diameter), each coated with a 1 nm Pd shell. (D) Plot of relative absorbed power as a function of CuO—Pd particle diameter.

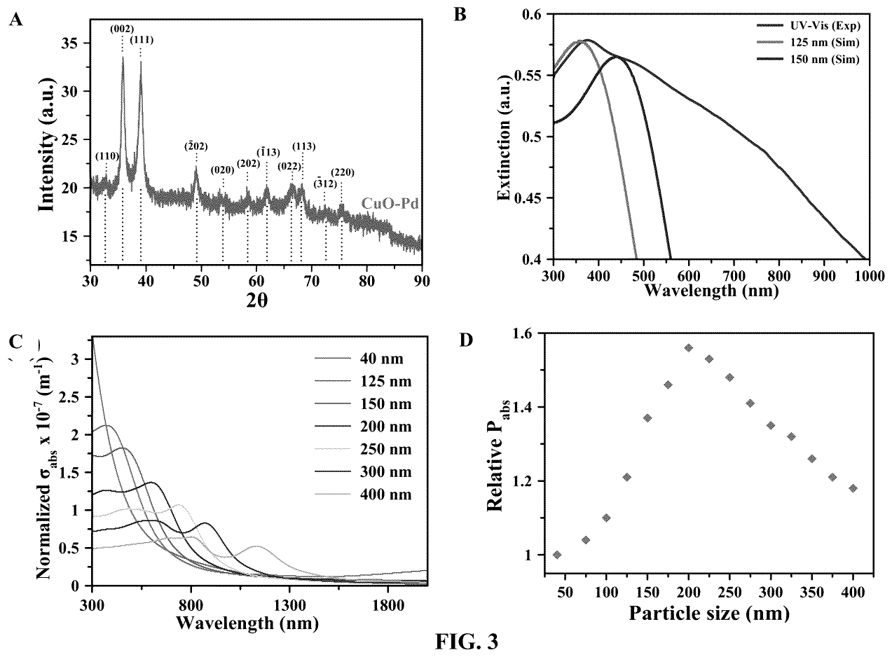

FIG. 4. Photocatalytic performance of CuO—Pd hybrid nanostructures. (A) Conversion of phenylacetylene (PA) under ~1 sun intensity illumination demonstrating accelerated reaction rates. (B) Comparison of temperature profiles under ~1 sun intensity light irradiation and external heating in the dark (Light-Off) conditions. (C) Rate enhancement due to light effect for 1 sun intensity. (D) Conversion of PA to DPDA under illumination (Light-On) and in the absence of light (Light-Off). (E) Temperature profile of the reaction system under visible-light irradiation. (F) Rate enhancement due to light effect for ~0.3 sun intensity.

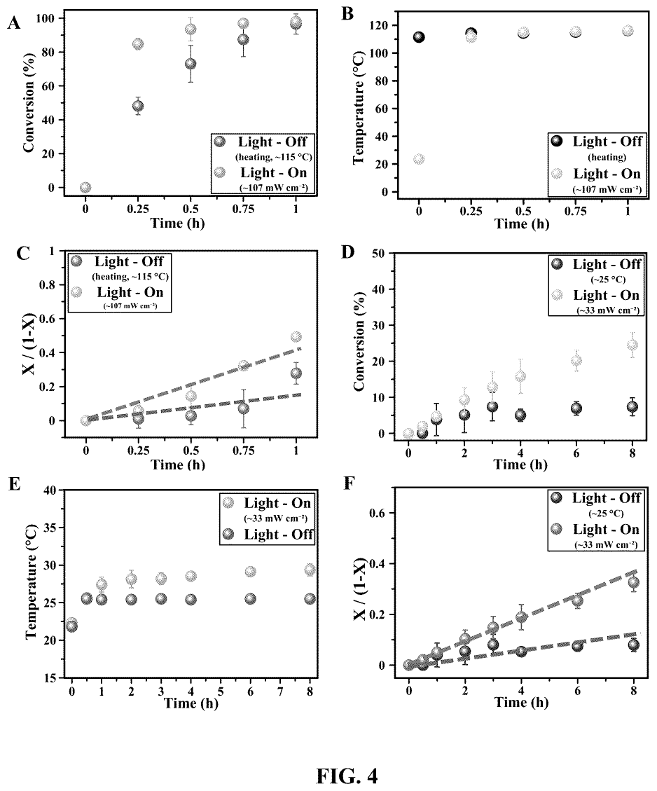

FIG. 5. Photocatalyst stability and base effect. (A) XRD patterns of CuO—Pd hybrid nanostructures before reaction and after photocatalytic oxidative C—C coupling of PA under Light-On conditions, confirming phase stability. (B) Conversion of PA to DPDA under high-intensity illumination in the presence and absence of K₂CO₃, showing negligible influence of the base on the reaction.

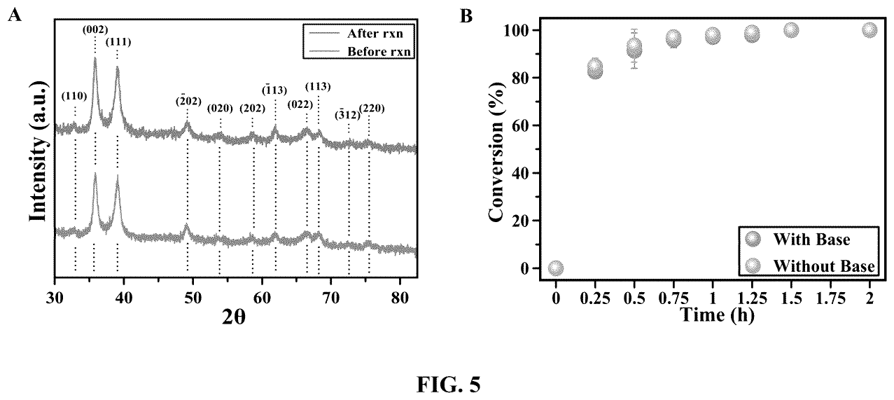

FIG. 6. Representative high-resolution transmission electron microscopy (HRTEM) image of CuO—Pd material showing Pd lattice fringes with interplanar spacing of 2.27 Å for Pd (111) and 2.0 Å for Pd (200) on CuO—Pd.

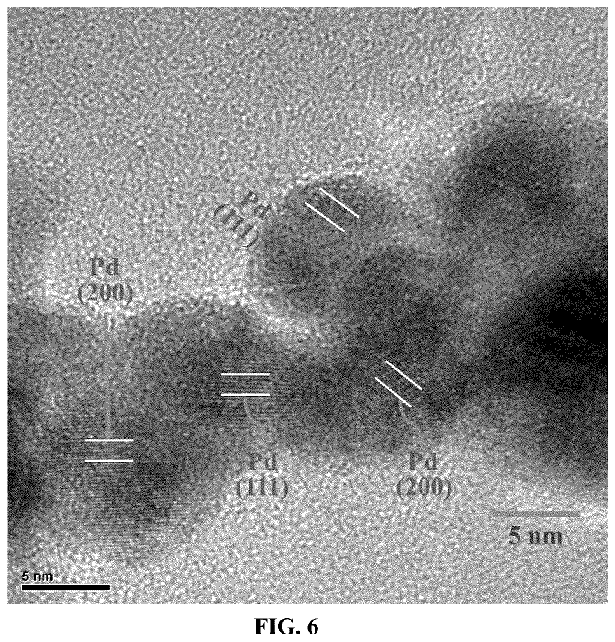

FIG. 7. FDTD simulations of CuO nanospheres. (A) Extinction cross sections as a function of incident light wavelength for particle diameters of 40-225 nm. (B) Extinction cross sections for particle diameters of 225-400 nm.

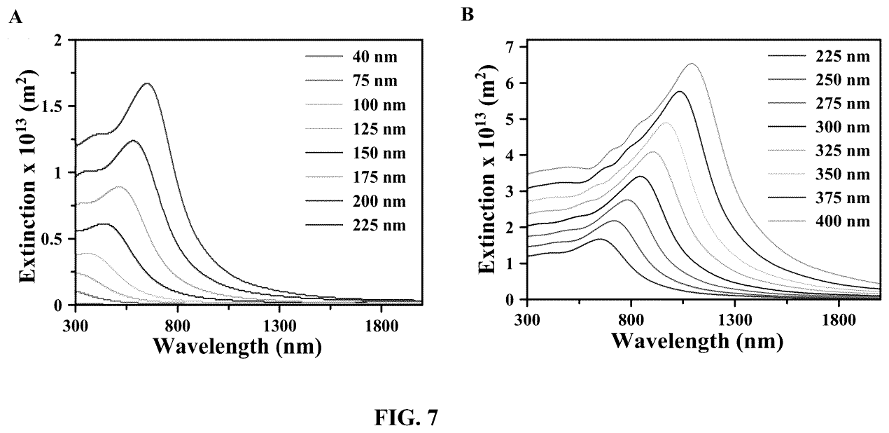

FIG. 8. Volume-normalized absorption cross section of CuO nanospheres. (A) FDTD-simulated shows the volume-normalized absorption cross sections of CuO nanospheres with diameters ranging from 40 to 225 nm. (B) FDTD-simulated show the volume-normalized absorption cross sections of CuO nanospheres with diameters ranging from 225 to 400 nm. Normalized σabs=Ratio of absorption cross section (σ_{abs}) to the volume of the nanosphere.

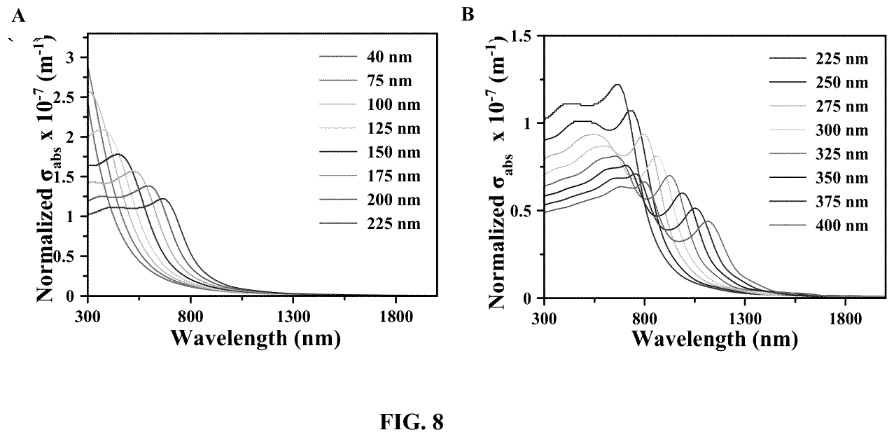

FIG. 9. Extinction spectra of CuO—Pd nanospheres with a 1 nm Pd shell. (A) Extinction cross sections as a function of incident light wavelength for particle diameters of 40-225 nm. (B) extinction cross sections for particle diameters of 225-400 nm.

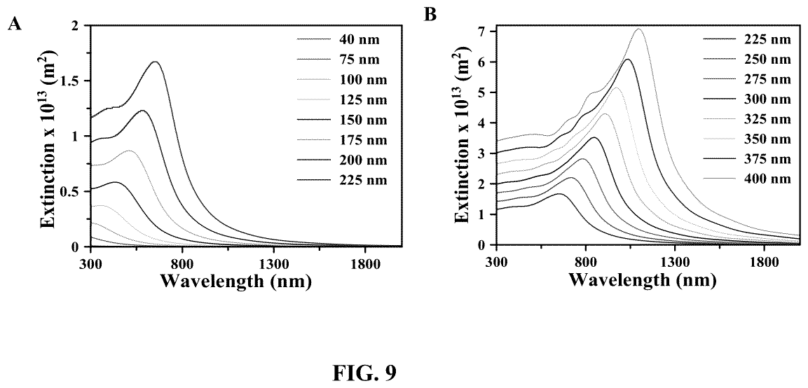

FIG. 10. Volume-normalized absorption cross section of CuO—Pd nanospheres. (A) FDTD-simulated normalized-extinction (normalized σ_{abs}) cross section of CuO—Pd nanospheres of different particle sizes ranging from 40 to 200 nm as a function of incident light wavelength. (B) FDTD-simulated normalized-extinction (normalized σ_{abs}) cross section of CuO—Pd nanospheres of different particle sizes ranging from 225 to 400 nm as a function of incident light wavelength.

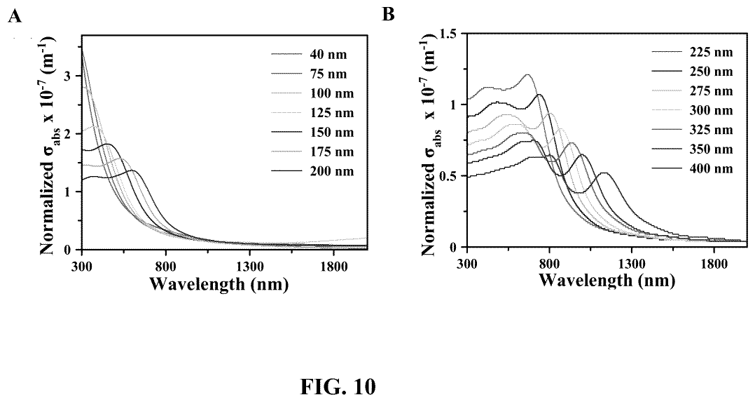

FIG. 11. Temperature profile of photocatalyzed oxidative carbon-carbon (C—C) coupling of phenylacetylene under Light-On conditions. The reaction is carried out at 107 mW cm⁻² (~1 sun intensity) light irradiation and atmospheric pressure.

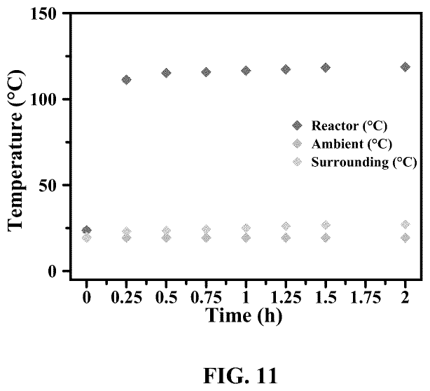

FIG. 12. SEM images of CuO—Pd nanocatalysts after the reaction. The morphological and structural properties of the nanocatalyst don't change significantly after the reaction.

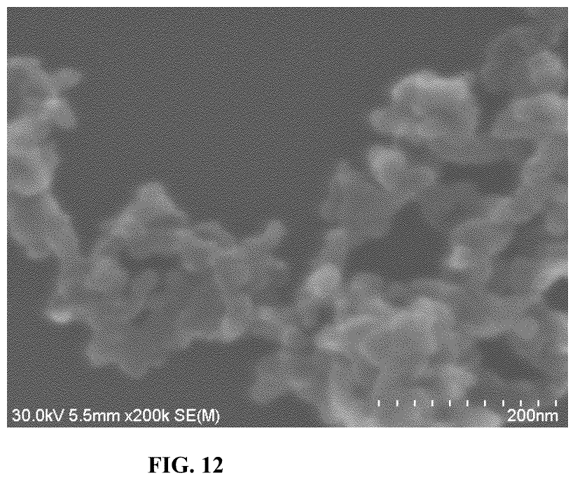

### DEFINITIONS

As used herein, the term “Mie resonator” refers to a dielectric particle that supports resonant electric and/or magnetic modes that enhance light absorption and local field intensity relative to a non-resonant particle of the same composition.

As used herein, the term “hybrid CuO—Pd photocatalyst” refers to a composite that includes CuO and Pd in interfacial electrical contact that establishes band bending at a junction between CuO and Pd.

As used herein, the term “base-free” refers to a reaction protocol in which no intentionally added inorganic base is present in the reaction mixture.

As used herein, the term “sun intensity” refers to a broadband illumination intensity calibrated relative to a solar-like spectrum at the reactor position, expressed in mW/cm².

As used herein, the term “terminal alkyne” refers to an alkyne that includes a hydrogen atom on the sp-hybridized carbon atom (RC≡CH).

As used herein, the term “oxidative homocoupling” refers to a reaction in which two equivalents of a terminal alkyne form a coupled diyne product in the presence of an oxidant.

As used herein, the term “oxygen-containing atmosphere” refers to a gas phase that includes molecular oxygen, and includes air, oxygen-enriched air, and oxygen diluted with an inert gas.

As used herein, the term “diyne” refers to a compound that includes two carbon-carbon triple bonds.

As used herein, the term “byproduct” refers to a product formed in addition to a target product in a chemical reaction.

As used herein, the term “broadband illumination” refers to illumination that spans a range of wavelengths that includes at least a portion of the visible region and at least a portion of the near-infrared region.

As used herein, the term “photon flux” refers to an incident photon rate per unit area over a specified wavelength range.

As used herein, the term “photothermal heating” refers to a temperature increase in a reaction system that results from optical absorption and conversion of incident radiation into heat.

As used herein, the term “photon-driven electronic effect” refers to a change in reaction rate or selectivity that results from photoexcited charge carriers interacting with adsorbed reactants at a catalyst surface under illumination at a matched bulk temperature condition.

As used herein, the term “Schottky junction” refers to a metal/semiconductor junction that establishes band bending and an internal electric field at the interface.

As used herein, the term “interfacial charge transfer” refers to transfer of charge carriers across an interface between two materials in electrical contact, including transfer between a semiconductor region and a metal region.

As used herein, the term “band bending” refers to a spatial variation in band-edge energy in a semiconductor near an interface arising from equilibration of Fermi levels across the interface.

As used herein, the term “nanocluster” refers to a nanoscale aggregate of atoms having a characteristic size of less than 10 nm.

As used herein, the term “shell” refers to a layer disposed over at least a portion of a particle core, where the layer has a thickness that is less than a characteristic dimension of the core.

As used herein, the term “effective optical size” refers to an apparent particle size that governs optical response in a medium, including effects from aggregation or clustering.

As used herein, the term “conversion” refers to the fraction or percentage of an identified reactant that is transformed into product(s) under specified reaction conditions.

As used herein, the term “selectivity” refers to the fraction or percentage of converted reactant that forms a specified product relative to all products formed.

As used herein, the term “rate enhancement factor” refers to a ratio of a rate constant (or fitted slope from a kinetic model) under a first condition to a rate constant (or fitted slope) under a second condition.

As used herein, the term “temperature-matched dark control” refers to a reaction performed without illumination at a temperature that matches a temperature measured during an illuminated reaction condition.

As used herein, the term “about” refers to a value that includes typical experimental uncertainty for the relevant measurement and, for numerical ranges, includes values within ±10% of the stated value unless a different tolerance is stated.

### DETAILED DESCRIPTION OF THE EMBODIMENTS

The embodiments described below illustrate representative implementations of the disclosed subject matter and do not limit the scope of protection. Features described in separate embodiments are combinable in additional embodiments.

In certain embodiments, a photocatalyst composition includes a CuO support having a characteristic dimension in a range from 10 nm to 500 nm and Pd disposed on the CuO support.

In certain embodiments, the CuO support includes quasi-spherical particles having a primary particle size in a range from 20 nm to 100 nm.

In certain embodiments, Pd is present as nanoclusters dispersed across the CuO surface, and the nanoclusters have an average size in a range from 0.5 nm to 5 nm.

In certain embodiments, Pd is present as an ultrathin shell over at least a portion of a CuO particle, and the shell thickness is in a range from 0.2 nm to 5 nm.

In certain embodiments, the effective optical size of the CuO—Pd catalyst in a reaction medium is governed by aggregation and falls in a range from 100 nm to 300 nm for spectral overlap with broadband illumination.

In certain embodiments, the CuO—Pd junction establishes a built-in field that drives directional charge separation under photoexcitation.

In certain embodiments, CuO nanoparticles are washed with acetone and dispersed in an alcohol solvent such as ethanol.

In certain embodiments, Pd is deposited onto dispersed CuO by contacting the CuO dispersion with an aqueous palladium salt solution under vigorous stirring for a short duration and then washing and drying the resulting solids.

In a representative embodiment, CuO dispersed in 3 mL ethanol is contacted with 21.33 mL of a 0.5 mM aqueous K₂PdCl₄ solution under vigorous stirring for 5-10 seconds, followed by washing with ethanol and/or acetone to yield CuO—Pd hybrid nanostructures.

In certain embodiments, a process forms a diyne product by oxidative homocoupling of a terminal alkyne in the presence of the CuO—Pd photocatalyst, an oxygen-containing atmosphere, and broadband illumination.

In certain embodiments, the terminal alkyne includes phenylacetylene (PA) and the diyne product includes 1,4-diphenylbutadiyne.

In certain embodiments, the oxygen-containing atmosphere includes air continuously flowed through a reaction vessel to maintain dissolved oxygen in the reaction medium.

In certain embodiments, the reaction medium includes N,N-dimethylformamide (DMF). In certain embodiments, the reaction medium includes a DMF/water mixture that includes about 80% DMF and about 20% water by volume.

In certain embodiments, the process operates under base-free conditions. In other embodiments, the process includes an inorganic base such as K₂CO₃ at an amount effective to increase reaction rate.

In certain embodiments, the light source provides broadband illumination spanning 300-2200 nm and the light intensity at the reactor position is in a range from 10 mW/cm² to 150 mW/cm².

In certain embodiments, the reaction vessel includes a round-bottom flask equipped with a condenser and a continuous air inlet, and the reaction mixture is stirred during illumination.

In certain embodiments, the terminal alkyne includes substituted aryl acetylenes. Representative substrates include 4-methylphenylacetylene, 4-methoxyphenylacetylene, 4-fluorophenylacetylene, 4-chlorophenylacetylene, 4-bromophenylacetylene, and 4-nitrophenylacetylene.

In certain embodiments, the hybrid Mie-resonator catalyst platform supports additional coupling reactions in which CuO provides light harvesting and Pd provides active surface sites. Reactions include Sonogashira coupling, Ullmann-type coupling, and oxidative C—H/C—H coupling under oxygen or air.

In certain embodiments, photon-driven electronic contributions are quantified by comparing (i) light-on operation and (ii) light-off operation at matched reaction temperature achieved by external heating.

In certain embodiments, conversion data are fitted to a second-order kinetic model and a rate enhancement factor is computed as a ratio of fitted rate constants for illuminated conditions relative to the corresponding dark control.

In certain embodiments, low-intensity illumination holds reaction temperature within about 25-28° C., or ambient temperatures, to suppress bulk heating contributions and isolate an electronic contribution.

In certain embodiments, an intensity distribution across a tray surface of a broadband light source is used to estimate and report illumination intensity at the reactor position as a function of radial position from the tray center (Table 1), consistent with tray-based solar-simulator intensity mapping techniques known in the art (see, e.g., Njie, D. N.; Rumsey, T. R., Drying Technology 1998, 16(1-2), 181-198).

In one embodiment, a photocatalytic reactor comprises a sealed cavity with chemically inert walls and a front-facing optical-grade quartz window to enable efficient broadband irradiation with sunlight.

In one embodiment, the reactor contains a magnetically stirred slurry of dielectric Mie-resonant metal oxide or hybrid metal/oxide photocatalysts. Reactants are introduced through a backside inlet while gaseous oxidants (e.g., O₂) are supplied to facilitate oxidative carbon-carbon coupling of terminal alkynes, with products exiting via an outlet port.

In one embodiment, the chemically inert walls include reflective inner surfaces that enhance an optical path length of broadband illumination within the sealed cavity, thereby enhancing light scattering and photon utilization, which is critical for Mie-resonant behavior. This configuration enables catalytic coupling under solar or simulated broadband light conditions.

In one embodiment, the chemically inert interior walls comprise a metal, glass, ceramic, or polymer material that is chemically resistant to the reaction medium and the terminal alkyne under the process conditions, and the interior walls comprise a reflective surface that increases an optical path length of the broadband illumination within the sealed cavity.

In one embodiment, the photocatalytic reactor further comprises one or more external reflectors positioned to direct solar radiation through the optical-grade quartz window and into the sealed cavity to irradiate a reaction mixture disposed within the sealed cavity.

In one embodiment, the one or more external reflectors comprise a non-concentrating reflector that redirects incident solar radiation toward the optical-grade quartz window without forming a focal region.

In one embodiment, the one or more external reflectors comprise a concentrating reflector configured to increase an intensity of solar radiation incident on the optical-grade quartz window relative to an ambient solar intensity at a location of the photocatalytic reactor.

In one embodiment, the sealed cavity comprises a reactor vessel positioned within the sealed cavity, wherein the reactor vessel contains a slurry comprising the photocatalyst composition and a reaction medium.

In one embodiment, the chemically inert interior walls comprise a reflective surface configured to reflect broadband illumination transmitted through the optical-grade quartz window and to redirect solar radiation to the reactor vessel positioned within the sealed cavity, thereby increasing an optical path length of the broadband illumination within the sealed cavity.

In one embodiment, the reflective surface comprises one or more reflective panels, curved reflective portions, baffles, or a combination thereof arranged to redirect incident solar radiation entering the sealed cavity through the optical-grade quartz window toward the reactor vessel.

In one embodiment, the reflective surface comprises a specular reflective surface, a diffuse reflective surface, or a combination thereof, and the reflective surface redirects solar radiation to the reactor vessel by one or more reflections within the sealed cavity.

In one embodiment, the concentrating reflector comprises a parabolic reflector dish and the photocatalytic reactor is positioned at or near a focal region of the parabolic reflector dish such that reflected solar radiation is directed through the optical-grade quartz window and into the sealed cavity.

In one embodiment, the concentrating reflector comprises a parabolic trough reflector and the photocatalytic reactor is positioned along a focal line of the parabolic trough reflector such that reflected solar radiation is directed through the optical-grade quartz window and into the sealed cavity.

In one embodiment, the one or more external reflectors comprise an array of planar mirrors oriented to direct solar radiation through the optical-grade quartz window and into the sealed cavity.

In one embodiment, the photocatalytic reactor operates as a continuous-flow reactor, wherein a reactant feed stream comprising the terminal alkyne and the reaction medium is delivered through a reactant inlet port located on a backside of the photocatalytic reactor and a product stream is removed through a product outlet port located on the backside of the photocatalytic reactor.

In one embodiment, the photocatalytic reactor operates as a batch reactor, wherein the sealed cavity further comprises a sealable access port located on a backside of the photocatalytic reactor, the sealable access port is configured to provide insertion of reactants into the sealed cavity before irradiation and removal of products from the sealed cavity after irradiation, and the sealable access port comprises a threaded cap, clamp, or gasketed closure that maintains a sealed condition during irradiation.

In one embodiment, the photocatalytic reactor further comprises a gas inlet configured to deliver an oxygen-containing atmosphere into the sealed cavity and a gas outlet configured to vent gas from the sealed cavity, wherein the gas inlet and the gas outlet are configured to maintain an oxygen-containing atmosphere during irradiation.

In one embodiment, the photocatalytic reactor comprises an internal mixing element comprising a magnetic stir bar or an impeller configured to mix a slurry comprising the photocatalyst composition and the reaction medium during irradiation to maintain suspension homogeneity.

In one embodiment, the photocatalytic reactor comprises a removable reaction vessel positioned within the sealed cavity, wherein the removable reaction vessel contains a slurry comprising the photocatalyst composition, the reaction medium, and the terminal alkyne, and the removable reaction vessel is configured for removal from the sealed cavity after irradiation.

In one embodiment, the reaction medium comprises a polar aprotic solvent selected from N,N-dimethylformamide (DMF), dimethyl sulfoxide (DMSO), N-methyl-2-pyrrolidone (NMP), N,N-dimethylacetamide (DMAc), acetonitrile, or a combination thereof.

In one embodiment, the reaction medium comprises an alcohol solvent selected from ethanol, methanol, 1-propanol, 2-propanol, 1-butanol, tert-butanol, or a combination thereof.

In one embodiment, the reaction medium comprises a solvent mixture comprising a polar aprotic solvent and water, wherein the water is present at a volume fraction from 1% to 50% relative to a total volume of the solvent mixture.

In one embodiment, the reaction medium comprises a solvent mixture comprising DMF and water.

In one embodiment, the reaction medium comprises a solvent mixture comprising DMSO and water.

In one embodiment, the reaction medium comprises a solvent mixture comprising an alcohol solvent and water.

In one embodiment, the reaction medium comprises a solvent mixture comprising a polar aprotic solvent and an alcohol solvent.

In one embodiment, the reaction medium comprises an ether solvent selected from tetrahydrofuran, 2-methyltetrahydrofuran, diethyl ether, or a combination thereof.

In one embodiment, the reaction medium comprises an aromatic solvent selected from toluene, xylene, anisole, or a combination thereof.

In one embodiment, the reaction medium comprises a solvent selected to solubilize the terminal alkyne and to maintain the photocatalyst composition as a slurry during irradiation.

In one embodiment, the reaction medium comprises a solvent selected to provide dissolved oxygen during irradiation under the oxygen-containing atmosphere.

In one embodiment, the process is performed using a first reaction medium comprising DMF and a second reaction medium comprising DMSO, and conversion and selectivity are determined for each reaction medium under otherwise matched irradiation conditions.

In one embodiment, the photocatalytic reactor comprising the parabolic reflector dish is operated with a reaction medium comprising a polar aprotic solvent and a slurry comprising the photocatalyst composition, wherein solar radiation directed by the parabolic reflector dish irradiates the slurry through the optical-grade quartz window.

In one embodiment, the batch reactor is operated with a reaction medium comprising ethanol or DMSO and a slurry comprising the photocatalyst composition, and the batch reactor is sealed to maintain the oxygen-containing atmosphere during irradiation.

### Methods

Catalyst preparation. CuO nanoparticles are washed with acetone and dispersed in ethanol. A palladium precursor solution (e.g., aqueous K₂PdCl₄) is added under vigorous mixing for a short duration to deposit Pd on CuO. The resulting solids are washed and dried to yield CuO—Pd hybrid nanostructures.

Catalyst characterization. UV-Vis extinction spectroscopy is performed on catalyst suspensions prepared in the reaction solvent (e.g., DMF/water). Electron microscopy (SEM, TEM, HAADF-STEM) and EDS mapping evaluate morphology and Pd distribution. XRD is collected over a 2θ range that includes 20°-100° to evaluate phase and stability before and after reaction.

Photoreactor operation. A round-bottom flask (e.g., 25 mL) containing a solvent (e.g., DMF or DMF/water), catalyst (e.g., 32 mg), and terminal alkyne (e.g., 0.91 mmol phenylacetylene) is illuminated with a broadband light source spanning 300-2200 nm. Air is flowed through the vessel via a condenser to supply oxygen. The reaction mixture is stirred and reflux conditions are maintained using water circulation.

Product analysis. Samples are withdrawn at intervals and analyzed by GC-MS using calibrated standards to quantify conversion and selectivity. In representative embodiments, phenylacetylene conversion yields 1,4-diphenylbutadiyne as the only detected product.

Temperature controls. Reactor and/or solution temperature is monitored during light-on experiments. Dark controls are performed at ambient temperature and at externally heated, temperature-matched conditions (e.g., about 115° C.) to separate electronic contributions from heating contributions.

Optical simulations. FDTD simulations compute extinction, scattering, and absorption cross sections for CuO and CuO—Pd nanospheres across 40-400 nm diameters over a 300-2200 nm wavelength range. Volume-normalized absorption cross sections and absorbed power are computed for constant catalyst mass and density to guide particle-size selection for spectral overlap with the illumination spectrum.

UV-Vis extinction spectra were collected using an Evolution 201 spectrophotometer with quartz cuvettes over a 200-1100 nm wavelength range with 1 nm resolution. Catalyst suspensions were prepared in a solvent mixture containing 80% N,N-dimethylformamide (DMF) and 20% deionized water by volume.

X-ray diffraction (XRD) data were collected using a Bruker AXS diffractometer with Cu Kα radiation (λ=1.5406 Å) operated at 40 kV and 40 mA. Data were acquired over a 2θ range of 20°-100°.

Scanning electron microscopy (SEM) imaging was performed using a Hitachi SU-70 microscope at an accelerating voltage of 30 kV and a working distance of 5 mm. For SEM sample preparation, catalyst powder was dispersed in 15 mL ethanol, 150 μL of that dispersion was diluted into 2 mL ethanol, and 10 μL of the diluted suspension was drop-cast onto a silicon wafer and dried prior to imaging.

Transmission electron microscopy (TEM) imaging was performed using an FEI Tecnai F20 microscope operated at 200 kV. The final ethanol suspension was drop-cast onto a carbon-coated TEM grid and dried under ambient conditions prior to imaging.

Catalytic conversion analyses were carried out using a Shimadzu GCMS-QP2020 NX system. Each product standard was calibrated in triplicate to support quantitative analyses.

Illumination intensity at the reactor position was adjusted by changing the distance between the broadband light source and the reactor. A distance-to-intensity mapping and a radius-dependent intensity distribution were used to relate lamp position to predicted intensity at the tray surface, which supports reproducible reporting of “sun intensity” across experiments.

TABLE 1

| Radiation intensity variation | |
|---|---|
| across the tray surface | |
| Radius | Predicted Intensity |
| (cm) | (mW/cm²) |
| 0 | 112 |
| 2.5 | 107 |
| 3.5 | 99.8 |
| 4.5 | 87.6 |
| 6 | 72.7 |
| 10 | 56.3 |
| 15 | 46 |
| 20 | 39.8 |
| 25 | 35.6 |
| 30 | 32.4 |
| 33 | 31.1 |
| 35 | 29.6 |
| 40 | 28 |

Finite-difference time-domain (FDTD) simulations were performed using the Lumerical FDTD package. Optical constants for Pd and CuO were taken from Palik. Perfectly matched layer (PML) boundary conditions were used in all spatial directions. Extinction, scattering, and absorption cross sections were computed as functions of wavelength using a total-field/scattered-field (TFSF) formulation with a Gaussian light source.

Absorbed power under an incident spectrum I0(λ) was computed using the following relationships:

$${P}_{a b s , N P} = \int {σ}_{a b s} ( λ ) {I}_{0} ( λ ) d λ$$

$$N = \frac{m}{ρ V}$$

$${P}_{a b s} = m ρ \int {σ}_{a b s}^{′} ( λ ) {I}_{0} ( λ ) d λ$$

$$Relative absorption power = \frac{{P}_{a b s}}{{P}_{abs , ref}}$$

For constant catalyst mass and density, a volume-normalized absorption cross section σ′abs(λ) was used to compare absorbed power across particle sizes. Relative absorbed power was reported as Pabs/Pabs,ref, where the reference corresponds to a defined particle size under the same spectral integration.

Photoreactor studies included four conditions: (i) Light-Off at ambient temperature, (ii) Light-On at ambient temperature under reduced intensity, (iii) Light-Off under external heating at a temperature matched to the light-induced temperature rise, and (iv) Light-On under higher intensity that produces a light-induced temperature rise. This framework isolates photon-driven electronic contributions relative to bulk heating at matched temperature.

$$X = \frac{( {C}_{A 0} - {C}_{A} )}{{C}_{A 0}}$$

$${C}_{A} = {C}_{A 0} ( 1 - X )$$

$$- \frac{d {C}_{A}}{d t} = k {C}_{A}^{2}$$

$$\frac{X}{( 1 - X )} = k {C}_{A 0} t$$

Control experiments performed under the manuscript reaction conditions show negligible product formation when pristine CuO is used without Pd, which establishes that Pd sites drive bond formation under those conditions.

Across the combined data set, CuO-only activity is condition dependent. Operating parameters that include solvent composition, base loading, catalyst size distribution, illumination intensity, and reactor configuration shift observed CuO-only conversion. The specification should distinguish regimes in which Pd is essential for detectable coupling product formation from regimes in which CuO-only catalysts show measurable activity.

TABLE 2
Rate enhancement for different light intensities.

| | kC_{A0} | Rate | | kC_{A0} | Rate |
|---|---|---|---|---|---|
| Room Temperature | (min⁻¹) | Enhancement | High Temperature | (min⁻¹) | Enhancement |
| Light - On | 0.0429 | 3.32 | Light - On | 0.6252 | 4.83 |
| (~ 32 mW/cm²) | | | (~ 107 mW/cm²) | | |
| Light - Off | 0.0129 | | Light - Off | 0.1293 | |
| (~ 25-30°° C.) | | | (~ 115-118°C) | | |

### Results

Catalyst structure and distribution. Electron microscopy shows quasi-spherical CuO—Pd nanostructures with primary particle sizes on the order of tens of nanometers and Pd dispersed as nanoclusters on the CuO surface. EDS mapping shows Pd distributed across the CuO particles. XRD patterns correspond predominantly to CuO and do not show distinct Pd peaks consistent with low Pd loading and small Pd domain size.

Light-harvesting behavior and design guidance. UV-Vis extinction spectra show a resonance feature consistent with dielectric Mie resonances. FDTD simulations across particle sizes yield volume-normalized absorption trends and an absorbed-power maximum as a function of particle size when integrated over a 300-2200 nm spectrum. The absorbed-power analysis identifies a size regime with improved spectral overlap with the illumination source.

Photocatalytic conversion under approximately 1 sun. Under broadband illumination at approximately 107 mW/cm², phenylacetylene conversion reaches complete conversion within about 1 hour with 1,4-diphenylbutadiyne as the only detected product. A temperature-matched dark control at about 115° C. reaches comparable conversion on a longer timescale. Kinetic fitting yields a rate enhancement factor of about 4.8 for illuminated conditions relative to the temperature-matched dark control.

Photocatalytic conversion under approximately 0.3 sun near ambient temperature. Under reduced illumination at approximately 33 mW/cm², reactor temperature stays within about 25-28° C. and conversion progresses over multi-hour timescales. Kinetic fitting yields a rate enhancement factor of about 3.3 for illuminated conditions relative to a dark ambient control, which isolates a photon-driven electronic contribution.

Base effect and waste profile. Under high-intensity illumination, base-free operation maintains high conversion and the byproduct profile corresponds to water as the stoichiometric byproduct of oxidative coupling of terminal alkynes under oxygen. Base-assisted operation increases rate under some conditions, and base-free operation reduces inorganic salt waste associated with base neutralization byproducts.

Stability. XRD patterns collected before and after reaction show no phase change, indicating that CuO remains stable under the tested reaction conditions. Post-reaction microscopy shows no significant morphology change at the evaluated scale.

What is claimed is:

1. A photocatalyst composition comprising:
(i) a cupric oxide (CuO) dielectric particle configured as a Mie resonator; and (ii) palladium disposed on the CuO dielectric particle, wherein the palladium comprises one or more members selected from the group consisting of (a) nanoclusters disposed on a surface of the CuO dielectric particle, (b) an ultrathin shell disposed over at least a portion of the CuO dielectric particle, and (c) a porous thin film disposed over at least a portion of the CuO dielectric particle, and wherein the photocatalyst composition is configured to catalyze oxidative carbon-carbon coupling of a terminal alkyne under broadband illumination and an oxygen-containing atmosphere.

2. A process comprising contacting a terminal alkyne with the photocatalyst composition of claim 1 in a reaction medium under an oxygen-containing atmosphere while irradiating the photocatalyst composition with broadband illumination at a reactor position, thereby forming a diyne product by oxidative homocoupling of the terminal alkyne.

3. A photocatalytic reactor comprising a sealed cavity comprising chemically inert interior walls, wherein the chemically inert interior walls comprise a reflective surface that increases an optical path length of broadband illumination and redirects solar radiation to a reactor vessel disposed within the sealed cavity; an optical-grade quartz window on a front side of the photocatalytic reactor, wherein the optical-grade quartz window transmits broadband illumination into the sealed cavity; a reactant inlet port and a product outlet port, wherein the reactant inlet port and the product outlet port are located on a backside of the photocatalytic reactor; a gas inlet configured to deliver an oxygen-containing atmosphere into the sealed cavity; a gas outlet configured to vent gas from the sealed cavity; and an internal mixing element comprising a magnetic stir element configured to mix a slurry disposed within the reactor vessel during irradiation.

4. The photocatalyst composition of claim 1, wherein the CuO dielectric particle has a primary particle size from 20 nm to 100 nm.

5. The process of claim 2, wherein an illumination intensity at a reactor position is about 33 mW/cm².

6. The photocatalyst composition of claim 1, wherein the palladium comprises an ultrathin shell having a thickness from 0.2 nm to 5 nm over at least a portion of the CuO dielectric particle.

7. The photocatalyst composition of claim 1, wherein the palladium comprises a porous thin film having a thickness from 0.2 nm to 5 nm over at least a portion of the CuO dielectric particle.

8. The process of claim 2, wherein an illumination intensity at a reactor position is from 10 mW/cm² to 150 mW/cm².

9. The process of claim 2, wherein the terminal alkyne comprises phenylacetylene and the diyne product comprises 1,4-diphenylbutadiyne.

10. The process of claim 2, wherein an illumination intensity at a reactor position is about 107 mW/cm².

11. The process of claim 2, wherein the reaction medium comprises a polar aprotic solvent selected from N,N-dimethylformamide (DMF), dimethyl sulfoxide (DMSO), N-methyl-2-pyrrolidone (NMP), N,N-dimethylacetamide (DMAc), acetonitrile, or a combination thereof.

12. The process of claim 2, wherein the reaction medium comprises an alcohol solvent selected from ethanol, methanol, 1-propanol, 2-propanol, 1-butanol, tert-butanol, or a combination thereof.

13. The process of claim 2, wherein the broadband illumination spans 300 nm to 2200 nm.

14. The process of claim 2, wherein the process is performed at 25-28° C. or at ambient conditions.

15. The process of claim 2, wherein the process is performed without an intentionally added inorganic base.

16. The process of claim 2, wherein the process is performed in the presence of an inorganic base comprising potassium carbonate.

17. The photocatalytic reactor of claim 3, further comprising one or more external reflectors positioned to direct solar radiation through the optical-grade quartz window and into the sealed cavity, wherein the one or more external reflectors comprise a non-concentrating reflector or a concentrating reflector.

18. The photocatalytic reactor of claim 17, wherein at least one external reflector comprises a concentrating reflector comprising a parabolic reflector dish, and wherein the photocatalytic reactor is positioned at or near a focal region of the parabolic reflector dish.

19. The photocatalytic reactor of claim 3, wherein the sealed cavity further comprises a sealable access port located on a backside of the photocatalytic reactor, wherein the sealable access port is configured to provide insertion of reactants into the sealed cavity before irradiation and removal of products from the sealed cavity after irradiation.

20. The process of claim 2, further comprising performing a temperature-matched dark control by conducting the process without the broadband illumination while externally heating the reaction medium to a temperature matched to a light-induced temperature rise during the process.
